(19) United States
(12) Patent Application Publication
(10) Pub. No.: US 2017/0173260 A1
(43) Pub. Date: Jun. 22, 2017

(54) INFUSION SYSTEM AND METHOD FOR CONTROLLING THE FLOW RATE OF LIQUID MEDICINE THEREOF

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Changfeng LI, Beijing (CN); Xue DONG, Beijing (CN); Haisheng WANG, Beijing (CN); Xiaochuan CHEN, Beijing (CN); Jiantao LIU, Beijing (CN); Yingming LIU, Beijing (CN); Shengji YANG, Beijing (CN); Weijie ZHAO, Beijing (CN); Lei WANG, Beijing (CN); Xiaoliang DING, Beijing (CN)

(21) Appl. No.: 15/124,329

(22) PCT Filed: Sep. 16, 2015
(86) PCT No.: PCT/CN2015/089767

§ 371 Date: Sep. 7, 2016

(30) Foreign Application Priority Data
May 19, 2015 (CN) 201510257057.4

(51) Int. Cl. A61M 5/172; A61B 5/00; A61B 5/024; A61M 5/142; A61M 5/168

(52) U.S. Cl. CPC A61M 5/1723; A61M 5/14244; A61M 5/16877; A61B 5/02438; A61B 5/4839; A61M 2205/3334; A61M 2205/3306; A61M 2205/502; A61M 2205/50; A61M 2205/3569; A61M 2205/583; A61M 2205/581; A61M 2230/005; A61M 2230/06

(57) ABSTRACT

This disclosure relates to the technical field of medical devices. Some embodiments provide an infusion system and a method for controlling a liquid flow rate of the infusion system, which can automatically adjusting the infusion flow rate according to the current physical condition of a patient. The infusion system comprises an infusion device and a monitoring device. The infusion device comprises an infusion tube for receiving liquid medicine from a liquid medicine container, and the infusion tube may be provided with an infusion flow rate regulating device for regulating the flow rate of the liquid medicine. The monitoring device may be configured for monitoring physiological parameters of the patient, and may control the infusion flow rate regulating device to regulate the flow rate of the liquid medicine according at least to the physiological parameter information of the patient.

### RELATED APPLICATIONS

The present application is the U.S. national phase entry of PCT/CN2015/089767 with an International filing date of Sep. 16, 2015, which claims the benefit of Chinese Application No. 201510257057.4, filed on May 19, 2015, the entire disclosures of which are incorporated herein by reference.

### TECHNICAL FIELD

The disclosure relates to the technical field of medical devices, and in particular to an infusion system and a method for controlling a flow rate of liquid medicine of the infusion system.

### BACKGROUND ART

All along, people's impression of adverse drug reactions has focused on the use of drugs. However, the information collected by China Food and Drug Administration shows that the adverse reactions caused by infusion shall also not be ignored.

The report recently released by China Food and Drug Administration shows that, since 2002 to the end of 2010, the National Centering for ADR (Adverse Drug Reaction) Monitoring received a total of 575 copies of Report Form for Suspected Medical Device Adverse Events concerning infusion pumps and injection pumps, including 359 copies on infusion pumps, and 216 copies on injection pumps. These reported cases mainly reflect infusion flow rate control abnormalities, failure to pump the liquid, crash, infusion line leaking and so on. Among the reports, there are 216 reports on infusion flow rate abnormalities, including 155 reports on infusion pumps (accounting for 43% of the total number of infusion pump reports), and 61 reports on injection pumps (accounting for 28% of the total number of injection pump reports).

For those intravenous infusion lovers in our country, such data is undoubtedly shocking. If the infusion flow rate is abnormal, it will directly affect the safety and effectiveness of the patient's medication. In general, the infusion flow rate should be determined according to the patient's age, condition, total amount of infusion, purpose of infusion and the nature of the drug, and so on. The common adult's flow rate is 40˜60 drops/min, whereas, for the children, the elderly or when infusing irritant drugs, the speed should not exceed 20˜40 drops/min. Patients with cardiopulmonary diseases and kidney diseases shall, in particular, strictly grasp the amount and flow rate of infusion.

At present, the liquid flow rate during an infusion needs to be adjusted manually, and cannot be adjusted to an appropriate flow rate according to the current state of the patient.

### SUMMARY

Some embodiments provide an infusion system capable of automatically adjusting the flow rate of the liquid medicine according to the current physical condition of the patient.

In order to achieve the above purpose, some embodiments adopt the following technical solutions:

An infusion system is provided, which comprises an infusion device and a monitoring device. The infusion device comprises an infusion tube for receiving liquid medicine from a liquid medicine container, and the infusion tube may be provided with an infusion flow rate regulating device for regulating a flow rate of the liquid medicine. The monitoring device may be configured for monitoring physiological parameters of a patient, and may control the infusion flow rate regulating device to regulate the flow rate of the liquid medicine according at least to the physiological parameter information of the patient.

Optionally, the monitoring device is a wrist monitoring device.

Optionally, the monitoring device comprises a physiological parameter monitoring module, a communication module and a processing module. The physiological parameter monitoring module is configured to monitor the patient's physiological parameters; the processing module is configured to obtain the liquid flow rate information at least according to the patient's physiological parameter information and send it to the communication module; the communication module is configured to send the liquid flow rate information to the infusion flow rate regulating device.

Further optionally, the physiological parameter monitoring module comprises a pulse monitoring module.

Further optionally, the pulse monitoring module includes a light source and an optical sensor.

Further optionally, the optical sensor is a photodiode.

Optionally, the monitoring device further comprises a storage module for storing the basic information of the liquid medicine. The processing module is configured to obtain the liquid flow rate information according to the physiological parameter information of the patient and the basic information of the liquid medicine obtained from the storage module and to send it to the communication module.

Further optionally, the basic information of the liquid medicine includes at least one of the name of the liquid medicine, the viscosity of the liquid medicine and the curative effect.

Optionally, the monitoring device further comprises a display module, which is configured for displaying the physiological parameter information of the patient.

Optionally, the infusion device further comprises a reminding device disposed at the liquid medicine container near the infusion tube. The reminding device is configured for sending a reminding signal to the communication module when the liquid level in the liquid medicine container is lower than the warning line. The communication module is further configured for sending the reminding signal to the processing module. The processing module is further configured for sending the liquid flow rate information that contains the liquid flow rate having been reduced to a preset value to the communication module according to the reminding signal, so that the infusion flow rate regulating device adjusts the liquid flow rate to the preset value after receiving the liquid flow rate information from the communication module.

Further optionally, the reminding device is further configured for sending alarm signals in the form of sound or light.

Some embodiments further provide a method for controlling the liquid flow rate of the infusion system, including: monitoring the patient's physiological parameters; controlling the liquid flow rate of infusion system at least according to the patient's physiological parameter information.

In some embodiments, some physiological parameters of the patient such as the pulse, blood pressure, heart rate may be monitored in real-time by the monitoring device so as to control in real time the infusion flow rate regulating device to regulate the liquid flow rate in the infusion process according to the monitored physiological parameter information, thereby the suitable liquid flow rate may be automatically regulated according to the patient's current physical condition, and it is ensured that the patient is in the best condition for treatment and the efficacy plays to the best. In addition, it can prevent patients from regulating the liquid flow rate of their own accord in the infusion process that may lead to an accident.

### BRIEF DESCRIPTION OF THE DRAWINGS

In order to more clearly illustrate some embodiments of this invention or the technical solutions in the prior art, the following are simple introductions to the drawings to be used in describing the embodiments and the prior art. It should be realized that the following figures are just some embodiments of this invention. A person having ordinary skill in the art may obtain other drawings according to these figures without having to do any creative work.

FIG. 1 is a schematic diagram 1 of an infusion system provided according to an embodiment;

FIG. 2 is a schematic diagram 2 of an infusion system provided according to an embodiment;

FIG. 3 is a schematic diagram 3 of an infusion system provided according to an embodiment;

FIG. 4 is a schematic diagram 4 of an infusion system provided according to an embodiment;

FIG. 5 is a schematic diagram 5 of an infusion system provided according to an embodiment;

FIG. 6 is a schematic diagram 6 of an infusion system provided according to an embodiment.

### REFERENCE SIGNS

10—infusion device; 101—liquid medicine container; 102—infusion tube; 103—infusion flow rate regulating device; 104—reminding device; 20—monitoring device; 201—physiological parameter monitoring module; 202—communication module; 203—processing module; 204—storage module; 205—display module; 30—wrist monitoring device.

### DETAILED DESCRIPTION OF THE INVENTION

To make a person having ordinary skill in the art better understand this objective, technical solution and advantages of some embodiments, the following will give a clear and complete description of the technical solutions of the embodiments in combination with the drawings of the embodiments. It should be realized that the embodiments described are only part rather than all of the embodiments of this invention. Based on the embodiments described and illustrated herein, all other embodiments obtained by a person having ordinary skill in the art without any creative effort involved shall fall within the scope protected by this invention.

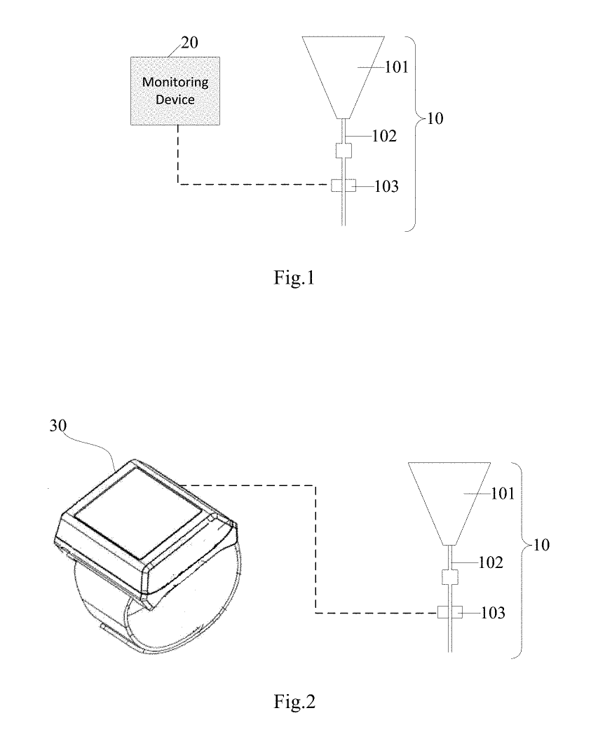

According to an embodiment, an infusion system is provided. As shown in FIG. 1, the infusion system may comprise an infusion device 10 and a monitoring device 20. The infusion device 10 may comprise an infusion tube 102 for receiving liquid medicine from a liquid medicine container 201. On the infusion tube 102 may be provided an infusion flow rate regulating device 103, the infusion flow rate regulating device 103 is configured to regulate the liquid flow rate. The monitoring device 20 may be configured to monitor the physiological parameters of the patient, and to control the infusion flow rate regulating device 103 to regulate the flow rate of the liquid medicine according at least to the physiological parameter information of the patient.

It should be noted that physiological parameters may be pulse, blood pressure, heart rate, blood flow, body temperature, etc., which can reflect a person's health indicators.

It should also be noted that in one implementation the monitoring device 20 may be configured to monitor the patient's physiological parameters, and at least according to the physiological parameter information of a patient control the infusion flow rate regulating device 103 to regulate the liquid flow rate, namely: the monitoring device 20 at least according to the patient's physiological parameter information sends a piece of control information to the infusion flow rate regulating device 103; the infusion flow rate regulating device 103 upon receiving the control information from the monitoring device 20 regulates the liquid flow rate.

Based on this, no limitation is made to the communication mode between the monitoring device 20 and the infusion flow rate regulating device 103. It may be communication in a wired manner or communication in a wireless manner.

In addition, no limitation is made to the structure of the infusion flow rate regulating device 103, as long as the liquid flow rate can be regulated according to the control information of the monitoring device 20.

As an example, the infusion flow rate regulating device 103 may comprise a box body, which includes a cavity that the infusion tube 102 can go through, and may further include a screw hole, the direction of the screw hole being perpendicular to the cavity direction along the infusion tube 102. In addition, the infusion flow rate regulating device 103 may also include a motor and a lead screw. The first end of the lead screw is connected with the motor, while the second end has a screw thread and is located at the screw hole. In this way, when the second end is in contact with the infusion tube 102 and extrudes the infusion tube 102, the flow rate of the infusion can be reduced. When the second end is gradually away from the infusion tube 102, the flow rate of the infusion can be increased. Wherein, by controlling the motor's rotation direction and number of turns, the lead screw is controlled to rotate clockwise or counter clockwise by a distance of movement to regulate the degree of extrusion of the infusion tube 102 and exercise control over the liquid flow rate. Here the motor may be a servo motor. The second end may also be provided with a cap whose surface is in the shape of an arc, in order to prevent the second end from destroying the infusion tube 102 when extruding the infusion tube 102.

In addition, the infusion flow rate regulating device 103 may also comprise a supporting rod, the supporting rod is used for supporting the lead screw and preventing the lead screw from pressuring the infusion tube 102 through the infusion flow rate regulating device 103 to make the infusion tube 102 break away from the liquid medicine container 101.

In the infusion system provided by an embodiment, some physiological parameters of the patient such as the pulse, blood pressure, heart rate and so on, can be monitored through the monitoring device 20 in real time, so as to control in real time the infusion flow rate regulating device 103 to regulate the liquid flow rate in the infusion process according to the monitored information of physiological parameters. Therefore, an appropriate liquid flow rate can be automatically regulated according to the patient's current physical condition, so as to ensure that the patient is treated in the best physical condition and make the pharmaceutical effects achieve best play. In addition, the patient can be prevented from regulating the liquid flow rate by himself or herself in the infusion process which may lead to an accident.

Optionally, as shown in FIG. 2, the monitoring device 20 is a wrist monitoring device 30.

The wrist monitoring device 30 can be worn on the wrist like a watch. On the one hand, it is easy to wear, and does not affect the patient's activities. On the other hand, the wrist of the human body has pulse and blood vessels, based on which the pulse, blood pressure, heart rate, blood flow rate and so on can be monitored.

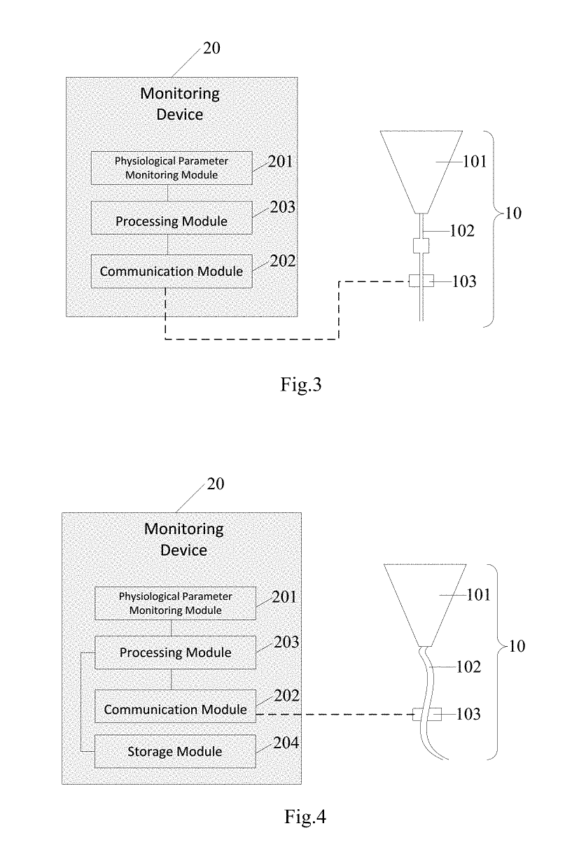

Further optionally, as shown in FIG. 3, the monitoring device 20 may include a physiological parameter monitoring module 201, a communication module 202, and a processing module 203. The physiological parameter monitoring module 201 is configured for monitoring the physiological parameters of the patient. The processing module 203 is configured to obtain the liquid flow rate information at least according to the physiological parameter information of the patient and send it to the communication module 202. The communication module 202 is configured for transmitting the liquid flow rate information to the infusion flow rate regulating device 103.

Here, for example, when the physiological parameter monitoring module 201 detects that the patient's pulse is greater than the standard value, based on which the processing module 203 may obtain the specific liquid flow rate. For example, the liquid flow rate may be decreased to 35 drops per minute, and the liquid flow rate information containing the flow rate of 35 drops per minute may be transmitted to the communication module 202, in order to make the communication module 202 send the liquid flow rate information to the infusion flow rate regulating device 103 to perform regulation of liquid flow rate.

Also for example, when the physiological parameters monitoring module 201 detects that the patient's pulse, blood pressure, heart rate and others are normal, based on which the processing module 203 can obtain the specific liquid flow rate. For example, the liquid flow rate is increased to 50 drops per minute, and the liquid flow rate information containing the flow rate of 50 drops per minute may be transmitted to the communication module 202, in order to make the communication module 202 send the liquid flow rate information to the infusion flow rate regulating device 103 to perform regulation of liquid flow rate.

The communication module 202, for example, may be a transceiver, and a wireless network can be used to realize the communication between the monitoring device 20 and the infusion flow rate regulating device 103. Or the communication module 202 can be Bluetooth. The Bluetooth wireless technology is adopted to achieve the communication between the monitoring device 20 and the infusion flow rate regulating device 103, namely it is via Bluetooth that the liquid flow rate information is sent to the infusion flow rate regulating device 103.

Further, taking into account that the heart rate, blood flow rate and so on can all be obtained by pulse calculation, therefore, according to an embodiment, the physiological parameter monitoring module 201 may include a pulse monitoring module. The pulse monitoring module may include a light source and an optical sensor. The light source may be a light emitting diode, which can emit infrared light or visible light.

This embodiment realizes the monitoring of the patient's pulse through a pulse monitoring module, the principle is: the variation of the intravascular hemoglobin absorbance is used to measure pulse, namely by emitting infrared light or visible light through a light source, the optical sensor receives the infrared light or visible light reflected or transmitted by the human body, then the pulse can be obtained by calculation.

Further, the optical sensor may be a photodiode.

A photodiode may be formed on a glass substrate or a silicon substrate. The photodiode may include an N-type silicon pattern layer, a P-type silicon pattern layer, and an intrinsic silicon pattern layer disposed between the N-type silicon pattern layer and the P-type silicon pattern layer.

This embodiment adopts a photodiode which can convert the optical signal reflected or transmitted by the human body into an electrical signal. Thus the embodiment can obtain the pulse and heart rate and so on by calculation through corresponding computational formulas according to variations in the electrical signal.

Optionally, as shown in FIG. 4, the monitoring device 20 may also include a storage module 204, which can be configured to store the basic information of the liquid medicine. The basic information of the liquid medicine may include at least one of the name of the liquid medicine, the viscosity of the liquid medicine and the curative effect. Based on this, the processing module 203 may be configured to, according to the patient's physiological parameter information and the basic information of the liquid medicine acquired from the storage module 204, obtain the liquid flow rate informatoin and send it to the communication module 202.

In this way, an appropriate liquid flow rate can be automatically regulated according to the patient's current physical condition and the liquid medicine's basic information to make the pharmaceutical effects achieve best play.

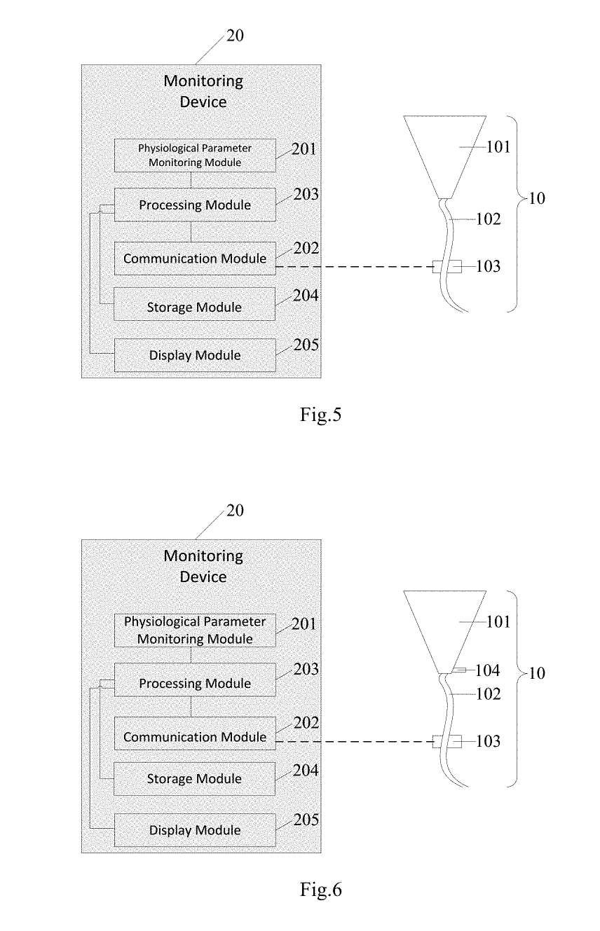

Optionally, as shown in FIG. 5, the monitoring device 20 may further include a display module 205, which can be configured to display the physiological parameter information of the patient.

To be specific, when the monitoring device is 20 is a wrist monitoring device 30, the display module 205 can be the front screen disposed on the wrist monitoring device 30, while other modules such as physiological parameter monitoring module 201, communication module 202, processing module 203, storage module 204 and so on may be integrated and disposed on a control circuit board inside the wrist monitoring device 30.

Displaying the physiological parameter information of a patient into the display module 205 can enable the patient to understand his or her physical condition so as to know what aspects he or she should pay attention to.

As shown in FIG. 6, in order to prevent accidents resulted from infusion at a previous liquid flow rate by an infusion device 10 when the liquid medicine in a liquid medicine container 101 has but a very small amount left, the infusion device according to an embodiment may further include a reminding device 104. The reminding device 104 may be arranged on the liquid medicine container 101 close to the infusion tube 102. The reminding device 104 may be configured for sending a reminding signal to the communication module 202 when the liquid level in the liquid medicine container 101 is lower than the warning line. On this basis, the communication module 202 may also be configured for sending the reminding signal to the processing module 203. The processing module 203 may be configured to, according to the reminding signal, send to the communication module 202 the liquid flow rate information that the liquid flow rate should be reduced to a preset value, so as to enable the infusion flow rate regulating device 103 to regulate the liquid flow rate to the preset value after receiving the liquid flow rate information from the communication module 202.

Further optionally, the reminding device 104 may also be configured to send an alarm signal in the form of sound or light.

In this way, either the patient or the medical staff upon hearing or seeing the alarm signal will check and change medicine or pull needles, to further ensure safety in the infusion process.

It should be understandable that the above is only an illustrative embodiment of this invention, and the scope of protection of this invention is not limited to this. Any technical personnel familiar with this technical field may easily think of variations or replacements within the technical scope disclosed by this invention, and these variations or replacements shall all covered by the scope of protection of this invention. Therefore, the scope of protection of this invention shall be subject to the scope of protection of the claims attached below.

It should be noted that the above embodiments only explain by examples according to the division of the various functional modules above, but in actual application, the above functions may be assigned according to actual needs to different functional modules to complete. The internal structure of the device can be divided into different functional modules to complete all or part of the functions described above. In addition, the function of one module above can be completed by a plurality of modules, and the functions of the multiple modules above can be integrated into one module to complete.

This application uses such words as “first”, “second”, “third” and so on. In the absence of any additional context, the use of such words is not intended to suggest the sequencing but is actually used for the purpose of identification. For example, the phrases “first edition” and “second version” do not necessarily mean the first version is exactly a first version, or a first version created before the second version or even requested or operated before the second version. In fact, these phrases are used to identify different versions.

In the Claims, no reference signs in parentheses should be construed as limiting the claims. The term “comprise” does not exclude the existence of elements or steps aside from the elements or steps listed in the claims. The word “a” or “an” in front of an element does not exclude the existence of multiple such elements. This invention can be realized by means of hardware including a plurality of separate components, and can be realized by properly programmed software or firmware, or by any combination of them.

In a claim for an apparatus or system that has listed several means, one or more of these means can be embodied in one and the same hardware item. The fact that a certain measure is recited in dependent claims that are different from each other does not indicate that the combination of these measures cannot be used to advantage.

1. An infusion system, comprising: an infusion device and a monitoring device,
the infusion device comprising an infusion tube for receiving liquid medicine from a liquid medicine container, the infusion tube being provided with an infusion flow rate regulating device for regulating a flow rate of the liquid medicine;
the monitoring device being configured for monitoring physiological parameters of a patient, and controlling the infusion flow rate regulating device to regulate the flow rate of the liquid medicine according at least to the physiological parameter information of the patient.

2. The infusion system of claim 1, wherein the monitoring device is a wrist monitoring device.

3. The infusion system of claim 1, wherein the monitoring device comprises a physiological parameter monitoring module, a communication module and a processing module;
the physiological parameter monitoring module being configured to monitor the patient's physiological parameters;
the processing module being configured to obtain the liquid flow rate information at least according to the patient's physiological parameter information and to send it to the communication module;
the communication module being configured to send the liquid flow rate information to the infusion flow rate regulating device.

4. The infusion system of claim 3, wherein the physiological parameter monitoring module comprises a pulse monitoring module.

5. The infusion system of claim 4, wherein the pulse monitoring module includes a light source and an optical sensor.

6. The infusion system of claim 5, wherein the optical sensor is a photodiode.

7. The infusion system of claim 3, wherein the monitoring device further comprises a storage module for storing basic information of the liquid medicine;
the processing module being configured to obtain the liquid flow rate information according to the physiological parameter information of the patient and the basic information of the liquid medicine obtained from the storage module and to send it to the communication module.

8. The infusion system of claim 7, wherein the basic information of the liquid medicine comprises at least one of the name of the liquid medicine, the viscosity of the liquid medicine and the curative effect.

9. The infusion system of claim 3, wherein the monitoring device further comprises a display module, which is configured for displaying the physiological parameter information of the patient.

10. The infusion system of claim 3, wherein the infusion device further comprises a reminding device disposed at the liquid medicine container near the infusion tube;
the reminding device being configured for sending a reminding signal to the communication module when a liquid level in the liquid medicine container is lower than a warning line;
the communication module being further configured for sending the reminding signal to the processing module;
the processing module being further configured for sending the liquid flow rate information that contains the liquid flow rate having been reduced to a preset value to the communication module according to the reminding signal, so that the infusion flow rate regulating device adjusts the liquid flow rate to the preset value after receiving the liquid flow rate information from the communication module.

11. The infusion system of claim 10, wherein the reminding device is further configured for sending alarm signals in the form of sound or light.

12. A method for controlling the liquid flow rate of the infusion system, including:
monitoring a patient's physiological parameters;
controlling a liquid flow rate of infusion system at least according to the patient's physiological parameter information.
